(19) United States
(12) Patent Application Publication
(10) Pub. No.: US 2022/0151907 A1
(43) Pub. Date: May 19, 2022

(54) PERSONAL CARE COMPOSITIONS

(71) Applicant: Lamberti SPA, Albizzate (IT)

(72) Inventors: Deborah D'ARRIGO, Busto Arsizio (IT); Thierry BOSSI, Azzio (IT); Simona ESPOSITO, Gallarate (IT); Rossana COLOMBO, Lugano (CH); Federico DE PELLEGRINI, Albizzate (IT); Giovanni FLORIDI, Novara (IT); Giuseppe LI BASSI, Gavirate (IT)

(73) Assignee: Lamberti SPA, Albizzate (IT)

(21) Appl. No.: 17/433,054

(22) PCT Filed: Mar. 13, 2020
(86) PCT No.: PCT/EP2020/056969

§ 371 Date: Aug. 23, 2021

(30) Foreign Application Priority Data
Mar. 15, 2019 (IT) 102019000003841

(51) Int. Cl. A61K 8/81; A61Q 19/00; A61Q 19/10; A61Q 5/00; C08F 220/18

(52) U.S. Cl. CPC A61K 8/8152; A61Q 19/00; A61Q 19/10; A61K 2800/48; C08F 220/1802; A61K 2800/262; A61Q 5/00

(57) ABSTRACT

Hydrophobically modified crosslinked acrylic polymers possessing good suspending and thickening capability and providing homogeneous and very clear formulation in the presence of surfactants and electrolytes, even at low pH, and aqueous personal care compositions comprising said hydrophobically modified crosslinked acrylic copolymers.

### TECHNICAL FIELD

The present invention relates to hydrophobically modified crosslinked acrylic polymers possessing good suspending and thickening capability and providing homogeneous and very clear compositions in the presence of surfactants and electrolytes, even at acidic pH.

The present invention also relates to personal care compositions and in particular to aqueous thickened compositions for use in personal care comprising said hydrophobically modified crosslinked acrylic polymers.

### PRIOR ART

It is known that a technical problem often encountered in the detergent & toiletries industries is to obtain stable and clear aqueous personal care compositions comprising surfactants, electrolytes and dispersed solid particles or insoluble liquid droplets.

In order to solve this problem, rheology modifiers (thickeners) are used in aqueous personal care products to increase their viscosity and suspension stability. An appropriate suspending ability is critical to achieving certain physical and aesthetic characteristics in an aqueous medium, such as the indefinite suspension of particles and/or of insoluble liquid droplets or the stabilization of gas bubbles within a liquid medium.

At the same time, the thickener should not influence the clarity of the said compositions.

In the field of aqueous personal care compositions, the thickeners are generally selected among crosslinked acrylic acids polymers (belonging to the class of crosslinked alkali swellable polyacrylates), guar gum and its derivatives, starch and its derivatives, crosslinked polyoxyethylene, carboxymethylcellulose, partially hydrolyzed crosslinked water swellable polymers, such as partially hydrolyzed polyacrylamides, and associative polyurethanes.

Crosslinked acrylic polymers and, in particular, hydrophobically modified crosslinked acrylic polymers, are often preferred.

Hydrophobically modified crosslinked acrylic polymers are associative polymers, containing pendant groups having both hydrophobic and hydrophilic regions. These groups are capable of interact with other groups in the polymer or with other ingredients of the compositions in which the polymer is present and can form non-specific “associations”, generally based on hydrophobic interactions, that usually enhance the viscosity of the compositions.

In the specialised literature, many products and methods are reported to regulate the rheological properties of different personal care compositions including the use of hydrophobically modified crosslinked acrylic polymers performing as thickeners at neutral and basic pH.

However, in the last years, there was an increasing interest, especially in the detergent & toiletries industries, to formulate personal care compositions with a pH in the range corresponding to the pH of the skin, i.e. between 4 and 6. The formulation of products in this range of pH allows to reduce the amount of preservatives, such as ascorbic acid and benzoic acid, and to increase their effectiveness. At the same time, as described by Wiechers J. W. in Cosm. & Toil. 123(XII), 61-70 (2008), a low pH is beneficial for repairing and maintaining the skin barrier tissue and helps the natural skin flora in excluding the colonization by microorganisms.

Thus, a number of patents regarding hydrophobically modified crosslinked acrylic polymers, showing the above mentioned properties also at acidic pH, has been filed.

For example, EP 2 935 375 describes an agent for obtaining a stable and clear acidic aqueous composition comprising suspended particles and a polymer constituted of: a) acrylic acid and/or methacrylic acid; b) an alkyl acrylate and/or methacrylate; c) an associative monomer corresponding to the following formula (I):

T-[(EO)ₙ(PO)_{n′}(BO)_{n″}]—Z (I)

in which: T represents one ethylenically unsaturated group, [(EO)ₙ(PO)_{n′}(BO)_{n″}] represents a polyalkoxylated chain (constituted of alkoxylated units, chosen among ethoxylated units (EO), propoxylated units (PO) and butoxylated units (BO)) and Z represents a fatty chain, linear or branched, of at least 16 carbon atoms; and d) at least one crosslinking monomer.

WO 2015/158668 relates to a method for making a direct emulsion of a copolymer in water, obtained by polymerization of: a) 10 to 80% by weight of methacrylic acid and of acrylic acid; b) 15 to 80% by weight of at least one non-ionic vinyl monomer; c) 0.05 to 9.5% by weight of 2-acrylamido-2-methylpropane sulfonic acid or a salt thereof; d) 0.5 to 30% by weight of at least one monomer containing at least one hydrophobic group; and e) 0.01 to 5% by weight of at least one crosslinking monomer; said polymerization being carried out in the presence of at least one surfactant. The direct emulsion of this copolymer imparts good clarity properties to aqueous formulations, even at acidic pH ranges, while maintaining desirable rheology properties of the formulations.

WO 2016/102790 discloses multiphase polymers comprising 45 to 95% by weight of a first polymer P1 and 5 to 55% by weight of a second polymer P2, said polymer P1 being obtained by polymerisation of a mixture of monomers comprising at least one anionic monomer (a), at least one nonionic hydrophobic monomer (b), at least one crosslinking monomer (c) and at least one associative monomer (d) having a hydrophobic hydrocarbon chain; and said polymer P2 being obtained by polymerisation of a mixture of monomers comprising at least one anionic monomer (a′), at least one nonionic hydrophobic monomer (b′), and at least one crosslinking monomer (c′).

US 2019/0002613 relates to hydrophobically modified alkali-swellable emulsion polymers that are useful as rheology modifiers and provide clear and stable suspensions; the polymers are characterized by the inclusion of an amphiphilic polyunsaturated macromonomer. In the examples US 2019/0002613 uses a single product, marketed and patented by Ethox Chemical LLC (E-Sperse®RS-1618, anionic polyethoxylated styrenated phenol containing two allyl groups from EP 2768904).

WO 2016/207554, WO 2017/207944 and WO 2017/207945 describe rheology modifying agents for acidic personal care formulations which are copolymers obtained by means of polymerization of at least one anionic ethylenically unsaturated monomer and at least one hydrophobic non-ionic ethylenically unsaturated monomer and at least one crosslinking monomer with specific formulas.

However, the personal care compositions thickened with the acrylic polymers disclosed in these patent applications, despite exhibiting good clarity and suspending properties in acidic pH range, tend to provide aqueous composition with high or very high Brookfield viscosities.

Unfortunately, thickeners with high viscosifying power can be difficult to handle, to dose and to dissolve/homogenize during manufacturing processes on a industrial scale. The personal care products obtained from these thickeners are typically difficult to apply and rinse away, especially if the the rheology modifier show a low shear thinning behaviour. High viscosities can also adversely affect packaging, dispensing, dissolution, and the sensory properties of the product.

Accordingly, there is a need for thickeners that do not significantly modify the ideal viscosity behaviour of a personal care composition, are able to suspend particles for extended periods of time, and show good clarity at pH ranging from about 4.0 to about 6.0.

We have now discovered that hydrophobically modified crosslinked acrylic polymers having the above described properties can be prepared by polymerizing ethyl acrylate and methacrylic acid with a fairly low amount of crosslinking monomer and an associative monomer containing a relatively short linear hydrophobic chain (mainly between C₈ and C₁₄).

These polymers possess remarkable suspending and clarity properties at acidic pH and show an optimal rheological behaviour in the presence of electrolytes and surfactants in comparison with the hydrophobically modified crosslinked acrylic copolymers of the prior art. The polymers of the present invention can beneficially thicken acidic aqueous personal care compositions to provide aesthetically smooth-textured products that flow smoothly and spread easily.

The polymers of the present invention are new and their properties are totally unexpected from the reading of the prior art.

WO 2016/207554 describes a hydrophobically modified crosslinked acrylic polymer containing methacrylic acid, ethyl acrylate, a crosslinking monomer and an associative monomer containing a relatively short branched hydrophobic chain (C12 oxo-alcohol). Unfortunately, compositions comprising the disclosed polymer in combination with surfactants show a high Brookfield viscosity and an insufficient trasmittance (clarity).

EP 2 935 375 describes comparative hydrophobically modified crosslinked acrylic copolymers containing methacrylic acid, ethyl acrylate, an associative monomer containing a linear C12 hydrophobic chain and an remarkably high amount of crosslinking monomer. Also in this case, the trasmittance at 500 nm of the compositions of these polymers with surfactants is extremely low.

### DESCRIPTION OF THE INVENTION

It is, therefore, an object of the present invention a hydrophobically modified crosslinked acrylic polymer obtainable by polymerization of a monomer composition consisting of:
a) from 50 to 68% by weight (wt %), preferably from 55 to 65% by weight, of ethyl acrylate;
b) from 25 to 40 wt %, preferably from 29 to 36 wt %, methacrylic acid;
c) from 0.005 to 2 wt %, preferably from 0.02 to 1 wt %, more preferably from 0.02 to 0.9 wt %, even more preferably from 0.02 to 0.45 wt %, of polyethylenically unsaturated monomers that are preferably selected in the group consisting of ethylene glycol di(meth)acrylate, triethylene glycol di(meth)acrylate, polyethylene glycol di(meth)acrylate, trimethylol propane tri(meth)acrylate and trimethylol ethane tri(meth)acrylate, pentaerythritol triallyl ether, allyl (meth)acrylate, trimethylol propane diallyl ether and diallyl maleate;
d) from 2.5 to 10 wt %, preferably from 3.5 to 8 wt % of a nonionic associative acrylic monomers having general formula I:

H₂C═C(R)—CO—O(CH₂CH₂O)ₙ—R′ I

wherein,
R is H or CH₃, the latter being preferred,
n is the average number of ethoxy groups and is at least 5, preferably at least 10, up to 40 or up to 70;
R′ is a linear alkyl chain having from 8 to 18 carbon atoms, preferably from 8 to 16 carbon atoms;
e) from 0 to 10 wt % of other monoethylenically unsaturated monomers;

with the proviso that at least 80 mole % of d) consists of monomers in which R′ is a linear alkyl chain having from 8 to 14 carbon atoms.

It is another object of the invention an aqueous personal care composition comprising from 0.1 to 10 wt %, preferably from 0.3 to 7 wt %, of said hydrophobically modified crosslinked acrylic polymer.

### DETAILED DESCRIPTION OF THE INVENTION

Preferably, the hydrophobically modified crosslinked acrylic polymer is obtained by polymerization of a monomer composition essentially consisting of the monomers from a) to d).

A key point in the preparation of hydrophobically modified crosslinked acrylic copolymer of the present invention is the presence in the monomer composition, of the nonionic associative acrylic monomer having general formula I.

Preferably, at least 90 mole %, more preferably at least 95 mole %, of d) consists of monomers having formula I in which R′ is a linear alkyl chain having from 8 to 14 carbon atoms.

In a preferred embodiment, about 100 mole % of d) consists of monomers having formula I, in which R′ is a linear alkyl chain having from 8 to 14 carbon atoms.

In a more preferred embodiment, about 100 mole % of d) consists of monomers having formula I, in which R′ is a linear alkyl chain having from 10 to 14 carbon atoms.

In the most preferred more preferred embodiment, about 100 mole % of d) consists of monomers having formula I, in which R′ is a linear alkyl chain having from 12 to 14 carbon atoms.

Specific examples of suitable monomers of formula I are decyl polyethoxy (meth)acrylate, dodecyl polyethoxy (meth)acrylate and tetradecyl polyethoxy (meth)acrylate, containing an average of from 5 to 70, preferably from 10 to 40, ethoxy groups, and mixtures of said monomers. The low amount of a polyethylenically unsaturated monomers, acting as polymer crosslinkers, is a further key point in the preparation of the hydrophobically modified crosslinked acrylic copolymer of the present invention.

The polyethylenically unsaturated monomers are chosen among known polyfunctional derivatives that undergo radical polymerization with (meth)acrylic monomers and contain two or more unsaturated moieties. Exemplary polyethylenically unsaturated monomers are (meth)acrylic compounds, for example di(meth)acrylate compounds such as ethylene glycol di(meth)acrylate, triethylene glycol di(meth)acrylate, 1,4-butylene glycol di(meth)acrylate, 1,6-hexanediol di(meth)acrylate, neopentyl glycol di(meth)acrylate, 1,9-nonanediol di(meth)acrylate, polyethylene glycol di(meth)acrylate; tri(meth)acrylate compounds such as, trimethylol propane tri(meth)acrylate and trimethylol ethane tri(meth)acrylate; tetra(meth)acrylate compounds such as ditrimethylol propane tetra(meth)acrylate and pentaerythritol tetra(meth)acrylate; hexa(meth)acrylate compounds such as dipentaerythritol hexa(meth)acrylate. Exemplary polyethylenically unsaturated monomers also include allyl compounds, such as allyl (meth)acrylate, diallyl itaconate, diallyl fumarate and diallyl maleate; polyallyl ethers of pentaerythritol such as pentaerythritol diallyl ether, pentaerythritol triallyl ether, and pentaerythritol tetraallyl ether, and combinations thereof; polyallyl ethers of trimethylolpropane such as trimethylolpropane diallyl ether, trimethylol propane triallyl ether, and combinations thereof. Other examples of polyethylenically unsaturated compounds include divinyl glycol, divinyl benzene, and methylenebisacrylamide.

The preferred polyethylenically unsaturated compounds are ethylene glycol di(meth)acrylate, triethylene glycol di(meth)acrylate, polyethylene glycol di(meth)acrylate, trimethylol propane tri(meth)acrylate and trimethylol ethane tri(meth)acrylate, pentaerythritol triallyl ether, allyl (meth)acrylate, trimethylol propane diallyl ether and diallyl maleate.

Diallyl maleate, pentaerythritol triallyl ether, allyl methacrylate and polyethylene glycol di(meth)acrylate are particularly preferred polyethylenically unsaturated monomers.

In a most preferred embodiment, the polyethylenically unsaturated monomers are chosen among allyl compounds. The preferred polyethylenically unsaturated monomers have a molecular weight below 400 daltons.

Beside the monomers from a) to d), the monomer composition of the disclosure can contain up to 10 wt %, preferably up to 5.0 wt %, of other monoethylenically unsaturated monomers e).

Advantageously, said monomers do not contain alkyl groups having more than eight carbon atoms, in particular C₈-C₂₄ alkyl groups; they can be chosen among ethylenically unsaturated monocarboxylic and dicarboxylic acids (and anhydrides thereof), ethylenically unsaturated sulfonic acids, (meth)acrylic acid esters of aliphatic C₁-C₈ alcohols, vinyl esters of C₁-C₈ aliphatic carboxylic acids, amides of (meth)acrylic acid, vinyl aromatic monomers and also vinyl chloride, vinylidene chloride, acrylonitrile.

Preferably they are chosen among ethylenically unsaturated monocarboxylic or sulfonic acids, (meth)acrylic acid esters of aliphatic C₁-C₈ alcohols, vinyl esters of C₁-C₈ aliphatic carboxylic acids, amides of (meth)acrylic acid, vinyl aromatic monomers and also vinyl chloride, vinylidene chloride, acrylonitrile; most preferably the monomers f), when present, are chosen among ethylenically unsaturated monocarboxylic acids, ethylenically unsaturated sulfonic acids and (meth)acrylic acid esters of aliphatic C₁-C₈ alcohols.

Suitable unsaturated monocarboxylic and dicarboxylic acids include acrylic acid, itaconic acid, maleic acid, maleic anhydride and combinations thereof. Exemplary unsaturated sulfonic acids include styrene sulfonic acid, acrylamidomethylpropane sulfonic acid (AMPS® monomer), vinyl sulfonic acid, allyl sulfonic acid and mixtures thereof.

Useful (meth)acrylic acid esters of aliphatic C₁-C₈ alcohols are, for example, methyl acrylate, propyl acrylate, isopropyl acrylate, butyl acrylate, hexyl acrylate, methyl methacrylate, ethyl methacrylate, isopropyl methacrylate, butyl methacrylate and mixtures thereof.

Examples of vinyl esters of C₁-C₈ aliphatic carboxylic acids are vinyl acetate, vinyl propionate, vinyl butyrate, vinyl isobutyrate, vinyl valerate, vinyl hexanoate, vinyl 2-methylhexanoate and combinations thereof.

Suitable vinyl aromatic monomers are styrene, alpha-methylstyrene, 3-methyl styrene, 4-methyl styrene, 4-propyl styrene, 4-tert-butyl styrene and mixtures thereof.

Acrylic acid and acrylamidomethylpropane sulfonic acid are the preferred monoethylenically unsaturated monomers f).

The hydrophobically modified crosslinked acrylic polymer of the present disclosure can be prepared by emulsion polymerization.

The emulsion polymerization techniques are well known in the art such as, for example, from U.S. Pat. Nos. 4,325,856; 4,654,397; and 4,814,373. Conventional surfactants may be used in emulsion polymerization, such as anionic and/or nonionic emulsifiers, for example, alkali metal or ammonium alkyl sulfates, alkyl sulfonic acids, fatty acids, and oxyethylated alkyl phenols. The amount of surfactant used is usually 0.1 to 6% by weight, based on the total weight of monomers. Thermal or redox initiation processes may be used. Conventional free radical initiators may be used such as, for example, hydrogen peroxide, t-butyl hydroperoxide, t-amyl hydroperoxide, alkali or ammonium persulfates, and azo initiators such as 4,4′-azobis(4-cyanopentanoic acid), and 2,2′-azobisisobutyronitrile (“AIBN”), typically at a level of 0.01% to 3.0% by weight, based on the weight of total monomer. Redox systems using the same initiators coupled with a suitable reductant such as, for example, sodium sulfoxylate formaldehyde, sodium hydrosulfite, isoascorbic acid, hydroxylamine sulfate and sodium bisulfite may be used at similar levels, optionally in combination with metal ions such as, for example, iron and copper, optionally further including complexing agents for the metal. Chain transfer agents such as mercaptans may be used to lower the molecular weight of the polymers. The monomer mixture may be added neat or as an emulsion in water. The monomer mixture may be added in a single addition or in multiple additions or continuously over the reaction period using a uniform or varying composition. The emulsion polymerization process may utilize a preformed seed emulsion polymer such as, for example, by adding 5% by weight (based on total weight of monomers) of the monomer mixture to the kettle and making it react previously. Techniques to reduce residual monomers such as, for example, subjecting the reaction mixture to steam stripping, hold times, and additional radical sources may be employed.

The hydrophobically modified crosslinked acrylic polymers of the present disclosure are generally supplied in their acidic form in emulsion; as they contain acidic groups, they need to be at least partially neutralized to the salt form to develop optimal viscosity increase in the personal care compositions.

An aqueous composition containing 2.25 wt % as active matter of hydrophobically modified crosslinked acrylic polymer of the invention, 9.0 wt % of sodium lauryl ether sulphate and 3.0 wt % of cocamidopropyl betaine and a preservative has advantageously a Brookfield RVT viscosity at pH 5.0, 25° C. and 20 rpm comprised between 500 and 6000 mPa*s, preferably between 2000 and 5000 mPa*s and shows a turbidity value below 50 NTU, preferably below 40 NTU.

The hydrophobically modified crosslinked acrylic polymer of the invention are useful as thickener and suspending agent for preparing stable and transparent aqueous personal care compositions. Usually, said compositions comprise from from 0.1 to 10 wt %, preferably from 0.3 to 7 wt %, more preferably from 0.5 to 4% wt % of said polymer.

An aqueous personal care composition is meant to be “transparent”, or “clear”, if it has a turbidity value below 50 NTU, preferably below 40 NTU. With the expression “aqueous personal care compositions” we mean the products normally used for personal detergence (such as: shampoos in general, and in particular 2-in-1 shampoos, baby shampoos, conditioning shampoos, moisturizing shampoos, temporary hair color shampoos, 3-in-1 shampoos, anti-dandruff shampoos, hair color maintenance shampoos, acid or neutralizing shampoos, salicylic acid shampoos; skin and body cleansers in general, and in particular shower gels, bath foams, facial cleansers, intimate cleansers, moisturizing body washes, antibacterial body washes; bath gels; hand soaps; bar soaps; body scrubs; bubble baths; facial scrubs; foot scrubs), hair care compositions (such as hair dyes, hair conditioners, hair creams and hair styling formulations), skin care compositions (such as skin lotions, balms and creams in general, and in particular alpha-hydroxy acid lotions and creams, beta-hydroxy acid creams and lotions, skin whiteners, self tanning lotions, sunscreen lotions, barrier lotions, moisturizers, vitamin C creams, antibacterial lotions and other moisturizing lotions and creams, liquid talc products, skin gels, for example facial masks, body masks, hydroalcoholic gels, body gels, sunscreen gels, make up foundations, sun care formulations, antiperspirants) and personal care products performing more than one of the above functions.

The aqueous personal care compositions can comprise from from 0.1 to 97% by weight of water, depending on the kind and the specific use of the composition.

The aqueous personal care compositions can also comprise from 0.1 to 60 wt %, preferably from 0.5 to 20 wt %, more preferably from 5 to 20 wt %, of a surfactant selected from anionic surfactants, amphoteric surfactants, cationic surfactants, zwitterionic surfactants, non-ionic surfactants, and mixture thereof.

According to an embodiment of the disclosure, the surfactant of the personal care composition of the invention is an anionic surfactant or a mixture of anionic surfactants.

Anionic surfactants include alkyl sulfates, alkyl ether sulfates, alkyl sulfonates, alkylamido sulfonates, alkylaryl sulfonates, alkylamido ether sulfates, alkylaryl polyether sulfates, monoglycerides sulfates, alpha-olefin sulfonates, paraffin sulfonates, alkyl and alkylaryl phosphates, alkyl ether and alkylaryl ether phosphates, alkyl sulfosuccinates, alkyl ether sulfosuccinates, alkylamide sulfosuccinate, alkylsulfoacetate, alkylsarcosinates, acylglutamate, alkyl carboxylates, fatty acids salts (soaps), fatty acyllactylates, alkyl ether carboxylates, anionic derivatives of alkyl polyglycosides, such as the citric, tartaric or sulfosuccinic ester of alkyl polyglucosides.

The amphoteric or zwitterionic surfactants which can be used in the compositions of the present disclosure are those which can be broadly described as derivatives of aliphatic amines or quaternary ammonium compounds, containing an anionic water-solubilizing group, e.g., a carboxylate, sulfonate, sulfate, phosphate or phosphonate group. Examples of amphoteric or zwitterionic surfactants include cocoamphocarboxypropionate, disodium lauroamphodiacetate, disodium caprylamphodiacetate, disodium cocoamphodipropionate, disodium lauroamphodipropionate, disodium caprylamphodipropionate, cocoamphocarboxy propionic acid, sodium cocoamphoacetate, disodium cocoamphodiacetate, sodium lauroamphoacetate, lauroamphodipropionic acid, cocoamphodipropionic acid, (C₈-C₂₀)alkyl betaines and (C₈-C₂₀)alkylamido(C₆-C₈) alkylbetaines, sultaines, (C₈-C₂₀)alkylamido(C₆-C₈) alkylsulfobetaines alkyl glycinates and alkyl carboxyglycinates.

Cationic surfactants useful in the compositions of the present disclosure contain amino or quaternary ammonium moieties which are positively charged when dissolved in the aqueous composition of the present invention. Examples of cationic surfactants are long-chain alkyl trimethyl ammonium chlorides, long-chain alkyl benzyl dimethyl ammonium chlorides, alkylamine hydrochlorides, alkylamine acetates and di(long-chain alkyl) dimethyl ammonium bromides.

In the case of anionic, cationic or amphoteric surfactants, the counter-ion can be monoatomic or polyatomic, inorganic or organic. Examples of counterions are: alkali metal, alkaline earth metal, transition metal, chloride (Cl⁻), bromide (Br⁻), iodide (I⁻), ammonium, pyridinium, triethanolaminium, methylsulfate.

Nonionic surfactants can be broadly defined as compounds containing a hydrophobic moiety and a nonionic hydrophilic moiety. Examples of the hydrophobic moiety can be alkyl, alkyl aromatic and aryl aromatic. Examples of hydrophilic moieties are polyoxyalkylenes, amine oxides, hydroxyl groups and alkanol amides. Examples of non ionic surfactants are fatty alcohols, alkoxylated fatty alcohols, alkoxylated fatty acids, glycerol alkyl esters, alkoxylated di- and tri-stiryl phenols, polyhydroxy fatty acid amides, sugar esters and polyesters, alkoxylated sugar esters, sorbitan and alkoxylated sorbitan fatty acid esters, block copolymers of polyethylene glycol and polypropylene glycol. Other examples of nonionic emulsifiers include alkyl polyglycosides, such as coco polyglucosides.

The aqueous personal care compositions of the present disclosure may further comprise a cosmetically acceptable medium. By “cosmetically acceptable medium” we mean a cosmetically acceptable solvent, which can be chosen among C1-C4 alcohols, such as ethanol, isopropanol, tert-butanol and n-butanol; polyols such as glycerol, propylene glycol and polyethylene glycols.

Additional ingredients of the aqueous personal care composition of the disclosure are those commonly known in the art and, by way of example, may be selected among, cationic polymers, other natural or synthetic thickeners, skin and hair care actives, particulate materials, perfumes and fragrances, silicones and water insoluble oily compounds, essential oils, dyes, pearlescent agents, opacifiers, scrubbing agents, enzymes, preservatives, disinfecting agents, antimicrobial additives, foaming agents, anti-foam agents, humectants, moisturizing agents, chelating agents, emollients, antiperspirant agents, conditioning agents, whitening agents, solvents and pH buffering means.

According to an embodiment of the disclosure, the aqueous personal care compositions additionally comprise at least one cationic polymer, useful for improving the cosmetic properties of skin or hair.

As disclosed herein, the therm “cationic polymer” means any polymer comprising at least one group that may be ionized into a cationic group. Among the cationic polymers that may be mentioned are, for example, polymers of the polyamine, polyamino amide and polyquaternium type.

Among the polymers, exemplary mention may be made of:
Acrylic copolymers and homopolymers;
Cellulose ether derivatives comprising quaternary ammonium groups;
Cationic guar gum derivatives;
Polymers of alkyldiallylamine or of dialkyldiallylammonium;
Polyquaternary ammonium polymers;
Quaternary polymer of vinylpirrolidone and vinylimidazole.

Examples of suitable cationic polimers are known under the INCI names: Polyquaternium-5, Polyquaterium-7, Polyquaternium-47, Polyqaternium-57, Polyquaternium-10, Guar Hydroxypropyltrimonium Chloride, Hydroxypropyl Guar Hydroxypropyltrimonium Chloride Polyquaternium-39, Polyquaternium-22, Polyquaternium-6, Polyquaternium-2, Polyquaternium-76, Polyquaternium 74, Polyquaternium-11, Polyquaternium-16, Polyquaternium-44, Polyquaternium-68, Cetrimonium Chloride, Behentrimonium Chloride, Steartrimonium Chloride, Acrylamidopropyl Trimonium Chloride/Acrylamide Copolymer.

The at least one cationic polymer may be present in the composition disclosed herein in a total amount ranging from 0.01 to 5% by weight relative to the total weight of the composition.

The aqueous personal care composition may also comprise one or more natural or synthetic thickeners other than the hydrophobically modified crosslinked acrylic polymer of the disclosure.

The aqueous personal care composition has preferably a Brookfield viscosity RVT viscosity at pH 5.0, 25° C. and 20 rpm comprised between 500 and 6000 mPa*s, preferably between 2000 and 5000 mPa*s.

The aqueous personal care composition of the present invention may further comprise at least one skin or hair care active (“actives”). Classes of suitable actives include, but are not limited to sunscreens, vitamins, peptides and peptide derivatives, sugar amines, oil control agents, flavonoid compounds, antioxidants, preservatives, phytosterols, protease inhibitors, tyrosinase inhibitors, anti-inflammatory agents, and mixtures thereof.

Herein, “sunscreen” is understood to include both sunscreen actives and UV light absorbers. The sunscreen may be organic or inorganic, and may be water-soluble, oil-soluble, a particulate material which is insoluble in either an oil or an aqueous phase, or a mixture thereof. In one embodiment the compositions of the present invention comprise a water-soluble and an oil-soluble sunscreen.

Particularly suitable sunscreen actives are known under the following INCI names: Benzophenone-3, Benzophenone-4, Bis-Ethylhexyloxyphenol Methoxyphenyl Triazine, Butil Methoxydibenzoylmethane, Diethylamino Hydroxybenzoil Hexyl benzoate, Disodium Phenyl Dibenzimidazole Tetrasulfonate, Drometrizole Trisiloxane, Methylene Bis-benzotriazolyl Tetaramethylbutylphenol, Terephthalylidene Dicamphor Sulfonic Acid, Zinc Oxide, 4-Methylbenzylidene Camphor, Polysilicone-15, Diethylhexyl Butamido Triazone, Ethylhexyl Dimethyl PABA, Ethylhexyl Methoxycinnamate, Ethylhexyl Salycilate, Ethylhexyl Triazone, Homomenthyl Salicylate, Isoamyl p-Methoxycinnamate, Octocrylene, Phenylbenzimidazole Sulfonic Acid, Titanium Dioxide, and mixtures thereof. Herein, “vitamins” means vitamins, pro-vitamins, and their salts, isomers and derivatives. The vitamins may include water soluble vitamins, for example, vitamin B compounds (including B3 compounds such as niacinamide; nicotinic acid, C1-C18 nicotinic acid esters, and nicotinyl alcohol; B6 compounds, such as pyroxidine; and B5 compounds, such as panthenol, or “pro-B5”); and vitamin C compounds, including ascorbyl esters of fatty acids, and ascorbic acid derivative, and mixtures thereof.

The vitamins also may include those exhibiting limited solubility in water, such as vitamin A compounds, and all natural and/or synthetic analogs of Vitamin A, including retinoids, carotenoids, and other compounds which possess the biological activity of Vitamin A; vitamin D compounds; vitamin E compounds, or tocopherol, including tocopherol sorbate, tocopherol acetate, other esters of tocopherol; vitamin K compounds; and mixtures thereof. In one embodiment, the aqueous personal care composition of the instant invention may comprise from about 0.0001% to about 10% by weight of the vitamin.

The aqueous personal care composition of the invention may comprise one or more peptides, for example, to aid in repair of skin, to aid in exfoliation, and to deliver other benefits to the skin. Herein, “peptide” refers to peptides containing ten or fewer amino acids, their derivatives, isomers, and complexes with other species such as metal ions (for example, copper, zinc, manganese, and magnesium). As used herein, peptide refers to both naturally occurring and synthesized peptides. In one embodiment, the peptides are di-, tri-, tetra-, penta-, and hexa-peptides, their salts, isomers, derivatives, and mixtures thereof.

The aqueous personal care composition may comprise a sugar amine, also known as amino sugars, and their salts, isomers, tautomers and derivatives. Sugar amines can be synthetic or natural in origin and can be used as pure compounds or as mixtures of compounds (e.g., extracts from natural sources or mixtures of synthetic materials).

The aqueous personal care composition may comprise one or more compounds useful for regulating the production of skin oil, or sebum, and for improving the appearance of oily skin. Examples of suitable oil control agents include salicylic acid, dehydroacetic acid, benzoyl peroxide, vitamin B3 compounds (for example, niacinamide), their isomers, esters, salts and derivatives, and mixtures thereof.

The aqueous personal care composition may comprise a flavonoid to provide anti-oxidation benefits. The flavonoid can be synthetic materials or obtained as extracts from natural sources, which also further may be derivatized.

The aqueous composition of the present invention may comprise non-vitamin antioxidants, preservatives, phytosterols and/or plant hormones, protease inhibitors, tyrosinase inhibitors, antiinflammatory agents and N-acyl amino acid compounds.

The aqueous personal care composition preferably also comprise a suspended particulate material. In one embodiment, the compositions may comprise from about 0.1 to about 10 wt % of a particulate material, and alternatively from about 1 to about 5 wt % of a particulate material. Suitable particulate materials include, but are not limited to almond meal, aluminum oxide, apricot seed powder, bismuth oxychloride, boron nitride, cellulose and cellulose derivatives, clay, calcium oxide, inorganic salts, for example salts of carbonates and chlorides, iron oxide, jojoba seed powder, mica, peach pit powder, pecan shell powder, polyethylene, polybutylene, polyisobutylene, polymethylstyrene, polypropylene, polystyrene, polyurethane, nylon, polytetrafluoroethylene, polyhalogenated olefins, pumice, rice bran, sericite, silk, synthetic hectorite, titanium dioxide, tricalcium phosphate, and mixtures thereof. Also useful are particles made from mixed polymers (e.g., copolymers, terpolymers, etc.). The polymeric and mixed polymeric particles can also optionally be cross linked with a variety of common crosslinking agents.

Other examples of useful particles include waxes and resins such as paraffins, carnauba wax, ozokerite wax, candellila wax, and urea-formaldehyde resins. When such waxes and resins are used herein it is important that these materials are solids at ambient and skin temperatures. Other examples of particulate materials useful in the present invention include colored and uncolored pigments, interference pigments, inorganic powders and organic powders other than those described above, composite powders, optical brightener particles, and mixtures thereof. The average size of such particulates in general may be smaller than the aforementioned particulate materials, ranging for example from about 0.1 microns to about 100 microns and can be added to the current compositions as a powder or as a pre-dispersion.

The aqueous personal care composition of the present invention may further comprise fragrance and perfume substances. Suitable fragrance and perfume substances include natural and synthetic fragrances and perfumes, and any other substances which emit a fragrance.

As natural fragrances, we mention those of vegetable origin, such as oil extracts from flowers, for example lily, lavender, jasmine, rose and the like; stems and leaves, such as geranium, petitgrain, peppermint; fruits, for example fennel, aniseed, coriander; fruit skin, such as bergamot, lemon, orange; herbs and grasses, such as sage, lemongrass and thyme; woods, such as pine tree, sandalwood, guaiacum wood, cedar, cinnamon; resins and balsam, for examples galbanum, frankincense and opopanax; and those of animal origin, such as musk, civet; and mixtures thereof. Examples of suitable synthetic fragrances and perfumes are hydrocarbons, alcohols, ethers, aldehydes, ketones, and aromatic esters, including benzyl acetate, phenoxyethyl isobutylate, linalyl acetate, dimethylbenzylcarbinyl acetate, phenylethyl acetate, benzyl formate, ethylmethylphenyl glycinate, allylcyclohexyl propionate, and benzyl salicylate; straight chain alkanals having 8 to 18 carbon atoms, lilial, citral, citronellal, hydroxycitronellal, and bougeonal; ionone compounds, α-isomethyl ionone, and methyl cedryl ketone; anethole, eugenol, isoeugenol, geraniol, lavandulol, nerolidol, linalool, and terpineol, terpenes, alpha-pinene and balsams; and mixtures thereof.

According to an embodiment of the disclosure, the personal care composition additionally comprise at least one silicone and/or water insoluble oil (oily compounds).

Silicone and oily compounds are often incorporated into personal care compositions for conditioning, especially hair and skin, and to improve or impart shine, gloss, water resistance or lubricity. These materials can also function as moisture barriers or protectants.

The silicone to be used according to this disclosure can be insoluble or soluble in water. Suitable water-insoluble, non-volatile silicones include amodimethicone, amodimethicone macroemulsions or microemulsions, polyalkylsiloxanes, poly-arylsiloxanes, polyalkylarylsiloxanes, polysiloxane gums, and polyethersiloxane copolymers. Preferred are high molecular weight polydimethylsiloxanes and other silicone materials such as dimethiconol, phenyldimethicone, polymethylphenyl polysiloxanes, organopolysiloxanes, alkoxysilicones, polydiorganosiloxanes, polydimethyl siloxane copolymers, and polyaminofunctional silicone. Water-insoluble silicones may also be considered oily conditioning agents.

Additional non-soluble, silicones which can be utilized include volatile silicones, for example cyclomethicone, or low viscosity polydimethylsiloxane.

Suitable water-soluble silicones include polyether/polysiloxane block copolymers, such as dimethicone copolyols, and derivatives thereof. The most preferred silicones used as an oily conditioning agent are polydimethylsiloxanes which have the CTFA designation of dimethicone, dimethiconol, and emulsions thereof.

Other suitable oily compounds to be used in the personal care compositions of the invention include, but are not limited to the following: mineral oils and saturated or unsaturated vegetable oils such as soybean oil, babassu oil, castor oil, cottonseed oil, Chinese tallow oil, crambe oil, perilia oil, Danish rapeseed, rice bran oil, palm oil, palm kernel oil, olive oil, linseed oil, coconut oil, sunflower oil, safflower oil, peanut oil, corn oil, sesame oil, and avocado oil, as well as petrolatum; d-limonene, and esters such as isopropylpalmitate, cetearyl octanoate, C₁₂₋₁₅ alkylbenzoate, octyl stearate, and other materials such as PPG-2 myristyl etherpropionate, and the like. The silicone or the oily compound, or the combinations thereof, can be present in a concentration of about 0.1 to about 20 wt %, more preferably of about 0.3 to about 7 wt %, and most preferably of about 0.5 to about 5 wt % on the composition.

The pH of the aqueous personal care compositions disclosed herein may range from 3 to 11, preferably from 3 to 7, and more preferably from 4 to 6, even more preferably it is about 5. Advantageously, they are characterized by having at pH 5.0 (and higher), a turbidity value below 50 NTU, preferably below 40 NTU.

The aqueous personal care composition may be prepared by simple dilution or dissolution of the acidic emulsion of the hydrophobically modified crosslinked acrylic polymer in a nearly neutral aqueous solution, accompanied by the addition of the surfactant(s), a pH corrector and any optional ingredient.

The surfactants, which are fundamental ingredients of the personal care composition of the invention, can be added after or before the acrylic polymer, but are preferably added after.

If needed, for the correction of the pH of the personal care composition, alkalis, such as sodium or potassium hydroxide, ethanolamine, ammonia, etc., can be added to the composition.

In some case, the pH of the obtained thickened composition may also be lowered, when and how advisable; common weak organic acids, such as citric acid, salicylic acid, lactic acid and the like, may be used.

### EXAMPLES

Characterization Methods

Brookfield RVT Viscosity (mPa*s) was determined at 25° C. and 20 rpm using a Brookfield® RVT viscometer.

The suspending performances of the acrylic polymers were determined using Florapearls® Jojoba STD Lapis beads, having an average particle size about 800-1200 μm (commercially available from Floratech). 10 beads were added to a 50 ml conical centrifuge vial (30×115 mm). The vial was subsequently filled with 40 ml of the acrylic polymers compositions comprising the surfactants, taking care that the beads remained at the bottom of the vial. Each vial was centrifuged at 500 rpm for 60 min and changes in the position of the beads were then recorded. The test is considered successful if all the beads remain on the bottom of the vial or within 20 mm from the bottom of the vial.

Turbidity (haziness), expressed as Nephelometric Turbidity Units (NTU), was determined using a nephelometric turbidimeter (2100P Turbidimeter, HACH USA) and HACH Portable Turbidimeter Sample Cells (60 mm×25 mm). A Turbidity Standard Kit (from HACH) was used as reference. The results are an average of four readings.

### Examples 1-20

In the Examples the following monomers or raw materials were used:
Methacrylic acid, (MAA);
Ethyl Acrylate, (EA);
Decyl Polyethoxy (25EO) Methacrylate, about 50 wt % active matter and 25 wt % methacrylic acid water solution, (DEM)
Dodecyl/Tetradecyl (75/25) Polyethoxy (23EO) Methacrylate, about 50 wt % active matter and 25 wt % methacrylic acid water solution, (LEM);
VISIOMER® C18 PEG 1105 MA, Stearyl Polyethoxy (25EO) Methacrylate from Evonik Performance Materials GmbH, (SEM);
Allyl Methacrylate, (ALMA);
Diallyl Maleate, (DAMA);
Pentaerythritol Triallyl Ether, (PETAE);
Polyethyleneglycol Diacrylate (MWₙ=750), (PEGDA);
Trimethylol Propane Triacrylate, (TM PTA);
Sodium Lauryl Sulfate, 29 wt % water solution, (SLS)
Sodium Lauryl Ether Sulfate, 27% wt % water solution, (SLES);
Cocamidopropyl Betaine, 28% water solution, (CAPB);
Sodium Benzoate;
Citric Acid (10% in peso water solution)

### Example 1

370.0 g of water and 8.0 g of sodium lauryl sulphate were charged in a 1000 ml polymerization reactor, equipped with stirrer, thermometer, heating and cooling devices, and heated to 85° C.

Aside, 200.0 g of water, 6.5 g of sodium lauryl sulphate, 195.0 g of ethyl acrylate, 100.0 g methacrylic acid, 25.0 g of DEM and 0.16 g of ALMA were charged into a stirred feed reactor and emulsified.

When the content of the polymerization reactor reached the temperature of about 85° C., a solution of 0.12 g of sodium persulfate (radical initiator) in 2 g of water were charged into the first reactor. Soon after, the monomers emulsion of the feed reactor was metered into the polymerization reactor over a period of 120 minutes. At the same time, a solution of 0.3 g of sodium persulfate in 55.0 g of water was metered into the polymerization reactor over a period of 180 minutes. The reaction mass was maintained under stirring for an additional hour at a temperature of about 85° C., then, cooled to 50° C.

Subsequently, 0.84 g of t-butyl hydroperoxide and 0.21 g of ascorbic acid dissolved in 15.0 g of water were metered into the first reactor over a period of 60 minutes. The polymeric emulsion was maintained under stirring for further 30 min at the temperature of 50° C.

Finally, the hydrophobically modified crosslinked acrylic polymer emulsion was cooled down and discharged.

Hydrophobically modified crosslinked acrylic polymer emulsions according to the invention and comparative were prepared following the procedure described above.

Tables 1 and 2 report the monomers used for the preparation of the polymers according to the invention of Examples 1-12 and 13-16, respectively, and their percent by weight based on the total weight of monomers.

Table 3 reports the monomers used for the preparation of the polymers of the comparative Examples 17-24 and their percent by weight based on the total weight of monomers.

TABLE 1

| | % MAA | % EA | % LEM | Crosslinker | % Cross. |
|---|---|---|---|---|---|
| Example 1 | 33.8 | 62.1 | 4.0 | ALMA | 0.05 |
| Example 2 | 33.8 | 60.9 | 5.1 | ALMA | 0.03 |
| Example 3 | 33.8 | 60.9 | 5.1 | ALMA | 0.05 |
| Example 4 | 33.8 | 62.1 | 4.0 | DAMA | 0.10 |
| Example 5 | 33.7 | 58.6 | 7.5 | DAMA | 0.06 |
| Example 6 | 33.6 | 60.6 | 5.1 | PEGDA | 0.68 |
| Example 7 | 33.6 | 59.6 | 6.2 | PEGDA | 0.50 |
| Example 8 | 33.5 | 61.6 | 4.0 | PEGDA | 0.85 |
| Example 9 | 33.8 | 60.9 | 5.1 | PETAE | 0.05 |
| Example 10 | 33.8 | 63.0 | 3.1 | PETAE | 0.07 |
| Example 11 | 33.5 | 61.6 | 4.0 | TMPTA | 0.80 |
| Example 12 | 33.5 | 60.5 | 5.1 | TMPTA | 0.79 |

TABLE 2

| | % MAA | % EA | % DEM | Crosslinker | % Cross. |
|---|---|---|---|---|---|
| Example 13 | 33.8 | 58.1 | 8.0 | DAMA | 0.08 |
| Example 14 | 33.8 | 58.1 | 8.0 | PETAE | 0.05 |
| Example 15 | 33.8 | 58.1 | 8.0 | ALMA | 0.03 |
| Example 16 | 33.6 | 57.8 | 8.0 | TMPTA | 0.62 |

TABLE 3

| | % MAA | % EA | % SEM | Crosslinker | % Cross. |
|---|---|---|---|---|---|
| Example 17* | 33.3 | 61.8 | 4.8 | DAMA | 0.14 |
| Example 18* | 33.4 | 62.9 | 3.6 | DAMA | 0.15 |
| Example 19* | 33.7 | 64.9 | 1.2 | DAMA | 0.15 |
| Example 20* | 33.9 | 66.0 | 0.0 | DAMA | 0.10 |
| Example 21* | 33.0 | 61.4 | 4.8 | PEGDA | 0.85 |
| Example 22* | 33.0 | 61.4 | 4.8 | TMPTA | 0.85 |
| Example 23* | 33.3 | 63.5 | 2.4 | TMPTA | 0.88 |
| Example 24* | 33.6 | 65.5 | 0.0 | TMPTA | 0.92 |

*Comparative

Characterization

The performances of the acrylic polymers of Examples 1-24 were evaluated on compositions containing 2.25 wt % as active matter of the acrylic polymer emulsions, surfactants and sodium benzoate, as preservative. The ingredients were added to deionized water in the order in which they were mentioned.

The final pH of the compositions was adjusted to about 5.0 by addition of citric acid. The compositions were carefully homogenized by stirring.

Table 4 reports the concentration as active matter of each ingredient of the compositions.

TABLE 4

| Ingredient | wt % |
|---|---|
| Polymer | 2.25 |
| SLES | 9.0 |
| CAPB | 3.0 |
| Sodium Benzoate | 0.4 |
| Citric Acid | to pH 5.0 |
| Water | to 100 |

Each composition was carefully centrifuged and allowed to stand overnight before characterization in order to remove all possible air bubbles.

Tables 5 and 6 report the Brookfield RVT viscosity, the result of the test about the suspending power, the turbidity of the compositions prepared with the acrylic polymer emulsions of Examples 1-24.

Tables 5 and 6 also show the Brookfield RVT viscosity (mPa*s) at 25° C. and 20 rpm of 1.0 wt %, as active matter, of the polymers of Examples 1-24 in deionized water and in 0.2 wt % NaCl water solution both at pH 7.5. The % viscosity drop (% Δ Viscosity) is reported in the same Tables.

TABLE 5

| | Surfactant Composition | | | | | |
|---|---|---|---|---|---|---|
| | pH 5.0 | | | Water Solution | | |
| | | Sus- | | pH 7.5 | | |
| | Tur- | pending | Vis- | Water | NaCl | % Δ |
| | bidity | Power | cosity | Viscosity | Viscosity | Viscosity |
| Example 1 | 39 | Pass | 4225 | 2150 | 670 | 68.8 |
| Example 2 | 27 | Pass | 4520 | 1845 | 738 | 60.0 |
| Example 3 | 35 | Pass | 4440 | 2140 | 690 | 67.8 |
| Example 4 | 39 | Pass | 4725 | 2580 | 782 | 69.7 |
| Example 5 | 31 | Pass | 4350 | 2130 | 790 | 62.9 |
| Example 6 | 39 | Pass | 4500 | 1860 | 628 | 66.2 |
| Example 7 | 32 | Pass | 3730 | 1850 | 698 | 62.3 |
| Example 8 | 39 | Pass | 4900 | 2560 | 690 | 73.0 |
| Example 9 | 33 | Pass | 4540 | 1930 | 660 | 65.8 |
| Example 10 | 34 | Pass | 4180 | 2050 | 546 | 73.4 |
| Example 11 | 45 | Pass | 4240 | 2120 | 930 | 56.1 |
| Example 12 | 44 | Pass | 4710 | 2350 | 568 | 75.8 |
| Example 13 | 36 | Pass | 4370 | 2690 | 580 | 78.4 |
| Example 14 | 35 | Pass | 4210 | 2100 | 550 | 73.8 |
| Example 15 | 34 | Pass | 4150 | 2110 | 550 | 73.9 |
| Example 16 | 48 | Pass | 4700 | 3210 | 632 | 80.3 |

TABLE 6

| | Surfactant | | | | | |
|---|---|---|---|---|---|---|
| | Compositions | | | | | |
| | pH 5.0 | | | Water Solutions | | |
| | | Sus- | | pH 7.5 | | |
| | Tur- | pending | Vis- | Water | NaCl | % Δ |
| | bidity | Power | cosity | Viscosity | Viscosity | Viscosity |
| Example 17* | 55 | Pass | 8850 | 10400 | 1520 | 85.4 |
| Example 18* | 59 | Pass | 9180 | 8100 | 618 | 92.4 |
| Example 19* | 55 | Pass | 6750 | 6200 | 1440 | 76.8 |
| Example 20* | 45 | Fail | 3320 | 2000 | 514 | 74.3 |
| Example 21* | 33 | Pass | 7880 | 11000 | 3725 | 66.1 |
| Example 22* | 52 | Pass | 8590 | 8200 | 1210 | 85.2 |
| Example 23* | 35 | Fail | 3130 | 3920 | 1410 | 64.0 |
| Example 24* | 50 | Fail | 2560 | 1120 | 310 | 72.3 |

*Comparative

The comparison between the values of Tables 5 and 6 demonstrates that the hydrophobically modified crosslinked acrylic polymers of the invention have superior performances compared to the acrylic polymers of the prior art.

1) A hydrophobically modified crosslinked acrylic polymer obtainable by polymerization of a monomer composition consisting of:
a) from 50 to 68% by weight (wt %) of ethyl acrylate;
b) from 25 to 40 wt % of methacrylic acid;
c) from 0.005 to 2 wt % of polyethylenically unsaturated monomers;
d) from 2.5 to 10 wt % of nonionic associative acrylic monomers having general formula I:
H₂C═C(R)—CO—O(CH₂CH₂O)ₙ—R′ I
wherein,
R is H or CH₃,
n is the average number of ethoxy groups and is at least 5,
R′ is a linear alkyl chain having 8 to 18 carbon atoms;
e) from 0 to 10 wt % of other monoethylenically unsaturated monomers chosen among ethylenically unsaturated monocarboxylic acids, ethylenically unsaturated sulfonic acids, (meth)acrylic acid esters of aliphatic C₁-C₈ alcohols, vinyl esters of C₁-C₈ aliphatic carboxylic acids, amides of (meth)acrylic acid, vinyl aromatic monomers and also vinyl chloride, vinylidene chloride, acrylonitrile,
with the proviso that at least 80 mol % of d) consists of monomers in which R′ is a linear alkyl chain having from 8 to 14 carbon atoms.

2) The hydrophobically modified crosslinked acrylic polymer of claim 1, obtainable by polymerization of a monomer composition consisting of:
a) from 55 to 65 wt % of ethyl acrylate;
b) from 29 to 36 wt % of methacrylic acid;
c) from 0.02 to 1 wt % of the polyethylenically unsaturated monomers;
d) from 3.5 to 8 wt % of nonionic associative acrylic monomers having general formula I;
e) from 0 to 10 wt % of the other monoethylenically unsaturated monomers.

3) The hydrophobically modified crosslinked acrylic polymer of claim 1, obtainable by polymerization of a monomer composition consisting of:
a) from 50 to 68% by weight (wt %) of ethyl acrylate;
b) from 25 to 40 wt % of methacrylic acid;
c) from 0.005 to 2 wt % of the polyethylenically unsaturated monomers;
d) from 2.5 to 10 wt % of nonionic associative acrylic monomers having general formula I.

4) The hydrophobically modified crosslinked acrylic polymer of claim 1, wherein 100 mole % of d) consists of monomers in which R′ is a linear alkyl chain having from 8 to 14 carbon atoms.

5) The hydrophobically modified crosslinked acrylic polymer of claim 1, wherein the polyethylenically unsaturated monomers c) are selected in the group consisting of ethylene glycol di(meth)acrylate, triethylene glycol di(meth)acrylate, polyethylene glycol di(meth)acrylate, trimethylol propane tri(meth)acrylate and trimethylol ethane tri(meth)acrylate, pentaerythritol triallyl ether, allyl (meth)acrylate, trimethylol propane diallyl ether and diallyl maleate;

6) The hydrophobically modified crosslinked acrylic polymer of claim 5 wherein the polyethylenically unsaturated monomers c) are selected in the group consisting of diallyl maleate, pentaerythritol triallyl ether, allyl (meth)acrylate and polyethylene glycol di(meth)acrylate.

7) The hydrophobically modified crosslinked acrylic polymer of claim 5, wherein the polyethylenically unsaturated monomers c) are chosen in the group which consist of pentaerythritol triallyl ether, allyl (meth)acrylate, trimethylol propane diallyl ether and diallyl maleate.

8) Aqueous personal care compositions comprising from 0.1 to 10 wt % of the hydrophobically modified crosslinked acrylic polymer of claim 1.

9) The aqueous personal care compositions of claim 8), comprising from 0.3 to 7 wt % of said hydrophobically modified crosslinked acrylic polymer.

10) The aqueous personal care compositions of claim 8), further comprising from 0.1 to 60 wt % of a surfactant selected from anionic surfactants, amphoteric surfactants, cationic surfactants, zwitterionic surfactants, non-ionic surfactants, and mixture thereof.

11) The aqueous personal care composition of claim 8 wherein said composition is a composition for personal detergence.

12) The aqueous personal care composition of claim 8 wherein said composition is a hair care compositions.

13) The aqueous personal care composition of claim 8 wherein said composition is a skin care composition.

14) The aqueous personal care composition of claim 8, wherein said composition is transparent.

15) Use of a hydrophobically modified crosslinked acrylic polymer according to claim 1 for the preparation of aqueous personal care compositions.
